(19) United States
(12) Patent Application Publication
(10) Pub. No.: US 2012/0142638 A1
(43) Pub. Date: Jun. 7, 2012

(54) COMBINATION OF ORGANIC COMPOUNDS

(76) Inventor: Randy Lee Webb, Flemington, NJ (US)

(21) Appl. No.: 13/371,882

(22) Filed: Feb. 13, 2012

Related U.S. Application Data
(63) Continuation of application No. 12/278,288, filed on Aug. 5, 2008, now abandoned.
(60) Provisional application No. 60/765,755, filed on Feb. 6, 2006.

(51) Int. Cl. A61K 31/675; A61K 31/519; A61K 31/437; A61K 31/4709; A61K 31/55; A61K 31/551; A61K 31/472; A61K 31/554; A61P 3/04; A61P 25/30; A61P 9/12; A61P 9/04; A61P 13/12; A61P 9/00; A61P 9/10; A61P 29/00; A61P 3/08; A61P 3/00; A61P 3/10; A61P 5/14; A61P 1/16; A61P 3/06; A61P 27/06; A61K 9/28; A61K 31/454

(52) U.S. Cl. 514/91; 514/326; 514/264.1; 514/303; 514/312; 514/212.07; 514/221; 514/307; 514/211.03; 427/2.14

(57) ABSTRACT

The present invention relates to a combination, such as a combined preparation or pharmaceutical composition, respectively, comprising a therapeutic agent acting on the renin-angiotensin system (RAS) or a pharmaceutically acceptable salt thereof and comprising at least one CB1 antagonist, or a pharmaceutically acceptable salt thereof.

The present invention furthermore relates to the use of such a combination for the prevention of, delay of progression of, treatment of diseases and disorders that may be modulated by action on the renin-angiotensin system (RAS), appetency disorders or substance abuse disorders.

This invention relates to a pharmaceutical combination comprising of cannabinoid receptor-1 (CB1) antagonists and a therapeutic agent acting on the renin-angiotensin system (RAS), in particular for the prevention, delay of progression or treatment of diseases and disorders that may be that may be modulated by action on the renin-angiotensin system (RAS), obesity, appetency disorders or substance abuse disorders.

Obesity and overweight greatly increase the risk of many diseases such as hypertension; type 2 diabetes; dyslipidemia; coronary heart disease; stroke; gallbladder disease; osteoarthritis; sleep apnea and other respiratory problems.

Weight loss is desirable in the case of diabetes, obesity and overweight individuals. Weight loss can help to prevent many of these harmful consequences, particularly with respect to diabetes and cardiovascular disease (CVD). Weight loss may also reduce blood pressure in both overweight hypertensive and non-hypertensive individuals; serum triglycerides levels and increases the beneficial high-density lipoprotein (HDL)-form of cholesterol. Weight loss also generally reduces somewhat the total serum cholesterol and low-density lipoprotein (LDL)-cholesterol levels. Weight loss may also reduce blood glucose levels in overweight and obese persons.

While weight loss and appetite control are desirable, it is hard to achieve. Many treatments for the management of appetite, overweight and obesity and the maintenance of weight loss exist. However, recidivism is rampant. Approximately 40 percent of women and 24 percent of men are trying to actively lose weight at any given time. These treatments include, but are not limited to, low-calorie diets and low-fat diets; increased physical exercise; behavioral therapies directed toward reducing food intake; pharmacotherapy; surgery; and combinations of the above.

The pharmacopeia of weight loss is relatively bare. A preferred way to reduce body weight is to reduce the appetite for foods and caloric beverages. Drugs such as sibutramine, dexfenfluramine, orlistat, phenylpropanolamine, phenteramine, or fenfluramine can facilitate weight loss in obese adults when used for prolonged periods. In general, however, the safety of long-term administration of pharmaco-therapeutic weight loss agents is unknown. For instance, recently due to concerns about valvular heart disease observed in patients, fenfluramine and dexfenfluramine have been withdrawn from the market. In the face of the slim pharmacopeia and the high prevalence of obesity and overweight, there is a need for new pharmaceutical methods and compositions to promote and maintain weight loss, for the treatment or prevention of diabetes, obesity, appetency disorders or substance abuse disorders.

High blood pressure becomes increasingly difficult to treat when patients present with additional co-morbidities such as diabetes, obesity or metabolic disturbances. To achieve target blood pressure goals in patients with coexistent risk factors or conditions, multi-drug therapy is often required. If blood pressure or other co-morbidities are inadequately modified, the patient is at greater risk of serious adverse events such as myocardial infarction, stroke and progressive organ damage.

Moreover, while renin angiotensin system (RAS) blockade, either with the use of angiotensin converting enzyme inhibitors (ACEi) or with angiotensin receptor blockers (ARBs) has proven to be a very effective means of lowering elevated blood pressure, many patients, for example, obese or overweight individuals, may require additional therapeutic interventions to achieve specific target blood pressure goals. Furthermore, obese or overweight patients or may need treatment with drugs designed specifically to interrupt key pathways contributing to this metabolic phenotype.

There is accumulating evidence that obese subjects have an increased risk of cardiovascular and metabolic diseases (Montani J P, Antic V, Yang Z. Pathways from obesity to hypertension: from the perspective of a vicious triangle. Internat J Obesity 26(Suppl 2):S28-S38, 2002). Also, a strong correlation between body weight and blood pressure has been demonstrated in humans (Jones D W, Kim J S, Andrew M E, Kim S J, Hong Y P. Body mass index and blood pressure in Korean men and women: the Korean National Blood Pressure Survey. J Hypertens 12:1433-1437, 1994). In short-term studies, it has been shown that weight loss in overweight or obese human subjects leads to a reduction in both systolic and diastolic blood pressure (Aucott L, Poobalan A, Smith W C S, Avenell A, Jung R, Broom J. Effects of weight loss in overweight/obese individuals and long-term hypertension outcomes. Hypertension 45:1035-1041, 2005). The precise mechanisms underlying this relationship are not known, however, a clear association between weight gain and activation of the sympathetic nervous system has been shown (Masuo K, Mikami H, Ogihara T, Tuck M L. Weight gain-induced blood pressure elevation. Hypertension 35:1135-1140, 2000). Increased sympathetic activity results in vasoconstriction and sodium retention, two factors that directly contribute to a rise in systemic blood pressure. In an animal model of diet-induced obesity in the dog, weight gain results in an increase in blood pressure, heart rate, and plasma insulin (Hall J E, Brands M W, Dixon W N, Smith M J Jr. Obesity-induced hypertension. Renal function and systemic hemodynamics. Hypertension 22:292-299, 1993). These results suggest that a similar cause and effect relationship may exist in animals and in humans and thus allows for the study of this set of conditions in appropriate animal species. Several additional factors also may contribute to the linkage seen between weight gain and blood pressure in animals and in man including leptin, free fatty acids, and insulin. Leptin and free fatty acids rise progressively with increasing adiposity and are released by visceral adipocytes. These mediators may act alone or in concert to increase sympathetic tone and vasoconstriction, thereby leading to an increase in blood pressure (Montani et al., 2002). Adipose tissue can be considered an endocrine organ, whereby release of leptin can have profound effects within the central nervous system to induce satiety and activate the sympathetic nervous system (Pantanetti P, Garrapa G G M, Mantero F, Boscaro M, Faloia E, Venarucci D. Adipose tissue as an endocrine organ? A review of recent data related to cardiovascular complications of endocrine dysfunctions. Clin Exper Hypertens 26(4):387-398, 2004). In obese humans, leptin is elevated in plasma yet these individuals do not appear to have a normal satiety response to this hormone. The concept of selective leptin resistance has been introduced to explain the phenomenon whereby the hypothalamus becomes unresponsive to the satiety effects of leptin but the central nervous system retains full reactivity to the stimulation of the sympathetic nervous system (SNS). Consequently, the obese or overweight phenotype lingers due to the inability of leptin to invoke a satiety response. Additionally, chronic over-activity of the SNS persists and leads to an increase in systemic blood pressure (Mark A L, Correia M L G, Rahmouni K, Haynes W G. Selective leptin resistance: a new concept in leptin physiology with cardiovascular implications. J Hypertens 20:1245-1250, 2002). Thus, human obesity is considered by some to be a leptin-resistant state. The model of the Agouti yellow obese mouse may mimic this phenotype (Correia M L G, Haynes W G, Rahmouni K, Morgan D A, Sivitz W I, Mark A L. The concept of selective leptin resistance. Diabetes 51:439-442, 2002).

Recently, it has been demonstrated that adipose tissue contains all of the components of the RAS (Goossens G H, Blaak E E, van Baak M A. Possible involvement of the adipose tissue renin-angiotensin system in the pathophysiology of obesity and obesity-related disorders. Obesity Reviews 4:43-55, 2003). Thus, the RAS contained in its entirety within the adipocyte may provide an important link between a major cardiovascular control system and obesity and obesity-related diseases. A high fat diet in rodents leads to increased generation of angiotensinogen and angiotensin II in adipocytes. Angiotensin II promotes adipocyte growth. Angiotensin II, either adipocyte-derived or formed in the plasma can have profound effects on fat cells directly or in distal cell types accessible from the circulation. Clearly, angiotensin II can result in a potent vasoconstrictor effect and sodium retention to increase arterial blood pressure. The findings relating components of the RAS within and/or released from adipocytes, have been equivocal in animal models and in humans (Engeli S, Schling P, Gorzelniak K, Boschmann M, Janke J, Ailhaud G, Teboul M, Massiera F, Sharma A M. The adipose-tissue renin-angiotensin-aldosterone system: role in the metabolic syndrome? Internat J Biochem Cell Biol 35:807-825, 2003).

Although the association between body weight and blood pressure is closely linked, the assignment of specific mechanisms underlying this relationship have been more difficult to prove since investigations have relied on several species, including man and the use of various animal models, cell systems and assay conditions.

Therefore, an object of the present invention is to provide more effective anti-obesity and/or compositions to treat cardiovascular disorders and new therapeutic methods with less or no side effects and lower toxicity for treating or preventing cardiovascular disorders, dyslipidemia or obesity, and conditions associated therewith.

It has now been found that a combination comprising at least one CB1 antagonist e.g., as defined below, and a therapeutic agent acting on the renin-angiotensin system (RAS) as co-agent, e.g., as defined below, has a beneficial effect and is useful in the treatment of obesity, appetency disorders, substance abuse disorders or conditions/disorders that might be may be modulated by action on the renin-angiotensin system (RAS).

Thus, the present invention relates to combinations, such as a combined preparation or pharmaceutical composition, respectively, comprising;
i) a therapeutic agent acting on the renin-angiotensin system (RAS) or a pharmaceutically acceptable salt thereof, and
ii) at least one CB1 antagonist, or a pharmaceutically acceptable salt thereof.

Preferably the present invention relates to a combination (pharmaceutical combination), such as a combined preparation or pharmaceutical composition, respectively, comprising;
i) a therapeutic agent acting on the renin-angiotensin system (RAS) or a pharmaceutically acceptable salt thereof, and
ii) at least one CB1 antagonist, or a pharmaceutically acceptable salt thereof.
   and at least one additional pharmaceutically acceptable carrier.

Preferably the combination is a pharmaceutical composition or a combined pharmaceutical preparation.

In this pharmaceutical composition, the combination partners (i) and (ii) can be administered together, one after the other or separately in one combined unit dosage form or in two separate unit dosage forms. The unit dosage form may also be a fixed combination.

The present invention is further related to the use of such a combination for the manufacture of a medicament for the prevention, delay of progression or treatment of diseases and disorders that may be modulated by action on the renin-angiotensin system (RAS), obesity, appetency disorders or substance abuse disorders.

The present invention is also directed to a method for the prevention of, delay of progression of, treatment of diseases and disorders that may be modulated by action on the renin-angiotensin system (RAS), obesity, appetency disorders or substance abuse disorders, comprising administering to a warm-blooded animal, including man, in need thereof an effective amount of the above combination.

Listed below are some of the definitions of various additional terms used herein to describe certain aspects of the present invention. However, the definitions used herein are those generally known in the art, e.g., hypertension, heart failure and atherosclerosis, and apply to the terms as they are used throughout the specification unless they are otherwise limited in specific instances.

The term “at least one CB1 antagonist” shall mean that in addition to the therapeutic agent acting on the renin-angiotensin system (RAS), one or more, for example two, furthermore three, active ingredients as specified according to the present invention can be combined. Preferably one or two CB1 antagonists are employed.

The term “renin-angiotensin system (RAS)” is meant to include the following phenomena: the secretion of renin by the kidney in response to a decrease in circulating volume and blood pressure; the cleavage of the substrate angiotensinogen to form the inactive decapeptide Angiotensin I; the conversion of Angiotensin I to the active octapeptide Angiotensin II by angiotensin converting enzyme (ACE); and the interaction of Angiotensin II with cellular receptors, such as the AT1 receptor, inducing vasoconstriction, the release of catecholamines from the adrenal medulla and prejunctional nerve endings, promoting the secretion of aldosterone and sodium reabsorption, and inhibiting renin release.

The term “therapeutic agents acting on the RAS” is meant to include any agents which block the renin-angiotensin system at any particular level. As a result the blood pressure and volume homeostasis can be positively regulated. Angiotensin II receptor blockers or Angiotensin II antagonists act on the RAS by inhibiting the interaction between Angiotensin II and the AT1 receptor. They are understood to be those active agents which bind to the AT₁-receptor subtype but do not result in activation of the receptor. ACE inhibitors block the conversion of Angiotensin I to Angiotensin II and potentiate bradykinin. Renin inhibitors act on the RAS at an earlier stage by blocking renin, thus, preventing the formation of Angiotensin I. As a result a smaller amount of Angiotensin II is produced.

The term “CB1 antagonist” is meant to denote an antagonist of the CB1 cannabinoid receptor. This is a compound which binds to the receptor and lacks any substantial ability to activate the receptor itself. An antagonist can thereby prevent or reduce the functional activation or occupation of the receptor by an agonist such as anandamide when the agonist is present. In some embodiments, the antagonist has an IC₅₀ from about 1 μM to about 1 nM. In other embodiments, the antagonist has an IC₅₀ of from about 0.1 μM to 0.01 μM, 1.0 μM to 0.1 μM, or 0.01 μM to 1 nM. In some embodiments, the antagonist competes with the agonist for binding to a shared binding site on the receptor.

The term “prevention” refers to prophylactic administration to healthy patients to prevent the development of the conditions mentioned herein. Moreover, the term “prevention” means prophylactic administration to patients being in a pre-stage of the conditions to be treated.

The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “synergistic”, as used herein, means that the effect achieved with the methods, combinations and pharmaceutical compositions of the present invention is greater than the sum of the effects that result from individual methods and compositions comprising the active ingredients of this invention separately.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

Diseases and disorders that may be modulated by action on the renin-angiotensin system (RAS) include but are not limited to

(a) hypertension, congestive heart failure, renal failure, especially chronic renal failure, restenosis after percutaneous transluminal angioplasty, and restenosis after coronary artery bypass surgery;
(b) atherosclerosis, eg., due to a reduction in oxidant stress, a direct effect on lipids or to an anti-inflammatory effect of one or all components of the combination,
(c) insulin resistance and syndrome X/metabolic syndrome, diabetes mellitus type 2, obesity, nephropathy, renal failure, e.g. chronic renal failure, hypothyroidism, survival post myocardial infarction (MI), coronary heart diseases, hypertension in the elderly, familial dyslipidemic hypertension, increase in the formation of collagen, fibrosis, eg., cardiac, renal or liver, remodeling (vascular) following hypertension and/or hyperlipidemia (antiproliferative effect of the combination which may be dependent or independent of an action on lipids), and vascular remodeling which may be, in part, due to an anti-inflammatory effect and all these diseases or conditions associated with or without hypertension;
(d) endothelial dysfunction with or without hypertension,
(e) hyperlipidemia, hyperlipoproteinemia, atherosclerosis and hypercholesterolemia,
(f) glaucoma
(g) isolated systolic hypertension (ISH),
(h) diabetic retinopathy and
(i) peripheral vascular disease.

The term “type 2 diabetes” including type 2 diabetes associated with hypertension refers to a disease in which the pancreas does not secrete sufficient insulin due to an impairment of pancreatic beta-cell function and/or in which there is to insensitivity to produced insulin (insulin resistance). Typically, the fasting plasma glucose is less than 126 mg/dL, while pre-diabetes is, e.g., a condition which is characterized by one of following conditions: impaired fasting glucose (110-125 mg/dL) and impaired glucose tolerance (fasting glucose levels less than 126 mg/dL and post-prandial glucose level between 140 mg/dL and 199 mg/dL). Type 2 diabetes mellitus can be associated with or without hypertension. Diabetes mellitus occurs frequently, e.g., in African American, Latino/Hispanic American, Native American, Native American, Asian American and Pacific Islanders. Markers of insulin resistance include HbAlC, HOMA IR, measuring collagen fragments, TGF-β in urine, PAI-1 and prorenin.

The term “hypertension” refers to a condition where the pressure of blood within the blood vessels is higher than normal as it circulates through the body. When the systolic pressure exceeds 150 mmHg or the diastolic pressure exceeds 90 mmHg for a sustained period of time, damage is done to the body. For example, excessive systolic pressure can rupture blood vessels anywhere, and when it occurs within the brain, a stroke results. Hypertension may also cause thickening and narrowing of the blood vessels which ultimately could lead to atherosclerosis.

The term “severe hypertension” refers to hypertension characterized by a systolic blood pressure of ≧180 mmHg and a diastolic blood pressure of ≧110 mmHg.

The term “pulmonary hypertension” (PH) refers to a blood vessel disorder of the lung in which the pressure in the pulmonary artery rises above normal level of ≦25/10 (especially primary and secondary PH), e.g., because the small vessels that supply blood to the lungs constrict or tighten up. According to the WHO, PH may be divided into five categories: pulmonary arterial hypertension (PAH), a PH occurring in the absence of a known cause is referred to as primary pulmonary hypertension, while secondary PH is caused by a condition selected, e.g., from emphysema; bronchitis; collagen vascular diseases, such as scleroderma, Crest syndrome or systemic lupus erythematosus (SLE); PH associated with disorders of the respiratory system; PH due to chronic thrombotic or embolic disease; PH due to disorders directly affecting the pulmonary blood vessels; and pulmonary venous hypertension (PVH).

The term “malignant hypertension” is usually defined as very high blood pressure with swelling of the optic nerve behind the eye, called papilledema (grade IV Keith-Wagner hypertensive retinopathy). This also includes malignant HTN of childhood.

The term “isolated systolic hypertension” refers to hypertension characterized by a systolic blood pressure of ≧140 mmHg and a diastolic blood pressure of <90 mmHg.

The term “familial dyslipidemic hypertension” is characterized by mixed dyslipidemic disorders. Biomarkers include oxidized LDL, HDL, glutathione and homocysteine LPa.

The term “renovascular hypertension” (renal artery stenosis) refers to a condition where the narrowing of the renal artery is significant which leads to an increase of the blood pressure resulting from signals sent out by the kidneys. Biomarkers include renin, PRA and prorenin.

The term “endothelial dysfunction” with or without hypertension refers to a condition in which normal dilation of blood vessels is impaired due to lack of endothelium-derived vasodilators. Biomarkers include CRP, IL6, ET1, BIG-ET1, VCAM and ICAM. Survival post-MI biomarkers include BNP and procollagen factors.

The term “diastolic dysfunction” refers to abnormal mechanical properties of the heart muscle (myocardium) and includes abnormal left ventricle (LV) diastolic distensibility, impaired filling, and slow or delayed relaxation regardless of whether the ejection fraction is normal or depressed and whether the patient is asymptomatic or symptomatic. Asymptomatic diastolic dysfunction is used to refer to an asymptomatic patient with a normal ejection fraction and an abnormal echo-Doppler pattern of LV filling which is often seen, for example, in patients with hypertensive heart disease. Thus, an asymptomatic patient with hypertensive left ventricular hypertrophy and an echocardiogram showing a normal ejection fraction and abnormal left ventricular filling can be said to have diastolic dysfunction. If such a patient were to exhibit symptoms of effort intolerance and dyspnea, especially if there were evidence of venous congestion and pulmonary edema, it would be more appropriate to use the term diastolic heart failure. This terminology parallels that used in asymptomatic and symptomatic patients with LV systolic dysfunction, and it facilitates the use of a pathophysiologic, diagnostic, and therapeutic framework that includes all patients with LV dysfunction whether or not they have symptoms (William H. Gaasch and Michael R. Zile, Annu. Rev. Med. 55: 373-94, 2004; Gerard P. Aurigemma, William H. Gaasch, N. Engl. J. Med. 351:1097-105, 2004).

The term “cardiac fibrosis” is defined as abnormally high accumulation of collagen and other extracellular matrix proteins due to the enhanced production or decreased degradation of these proteins. Biomarkers include BNP, procollagen factors, LVH, AGE RAGE and CAGE.

The term “peripheral vascular disease” (PVD) refers to the damage or dysfunction of peripheral blood vessels. There are two types of peripheral vascular diseases: peripheral arterial disease (PAD) which refers to diseased peripheral arteries and peripheral venous disorders, which can be measured by an ankle brachial index. PAD is a condition that progressively hardens and narrows arteries due to a gradual buildup of plaque and refers to conditions that effect the blood vessels, such as arteries, veins and capillaries, of the body outside the heart. This is also known as peripheral venous disorder.

The term “atherosclerosis” comes from the Greek words athero (meaning gruel or paste) and sclerosis (hardness). It's the name of the process in which deposits of fatty substances, cholesterol, cellular waste products, calcium and other substances build up in the inner lining of an artery. This buildup is called plaque. It usually affects large and medium-sized arteries. Some hardening of arteries often occurs when people grow older. Plaques can grow large enough to significantly reduce the blood's flow through an artery. But most of the damage occurs when they become fragile and rupture. Plaques that rupture cause blood clots to form that can block blood flow or break off and travel to another part of the body. If either happens and blocks a blood vessel that feeds the heart, it causes a heart attack. If it blocks a blood vessel that feeds the brain, it causes a stroke. And if blood supply to the arms or legs is reduced, it can cause difficulty walking and eventually gangrene.

The term “coronary arterial disease” (CAD) also refers to a condition that progressively hardens and narrows arteries due to a gradual buildup of plaque and refers to conditions that effect the blood vessels such as arteries within the heart. CAD is peculiar form of atherosclerosis that occurs in the three small arteries supplying the heart muscle with oxygen-rich blood. Biomarkers include CPK and Troponin.

The term “cerebrovascular diseases” comprise stroke conditions, such as embolic and thrombotic stroke; large vessel thrombosis and small vessel disease; and hemorrhagic stroke.

The term “embolic stroke” refers to a condition characterized by the formation of blood clots, e.g., in the heart, when clots travel down through the bloodstream in the brain. This may lead to a blockade of small blood vessels and causing a stroke.

The term “thrombotic stroke” refers to a condition where the blood flow is impaired because of a blockade to one or more of the arteries supplying blood to the brain. This process normally leads to thrombosis causing thrombotic strokes. Biomarkers include PAI 1, TPA and platelet function.

The term “metabolic syndrome” (Syndrome X) refers to an overall condition characterized by three or more of the following criteria:
1. abdominal obesity: waist circumference >102 cm in men, and >88 cm in women;
2. hypertriglyceridemia: >150 mg/dL (1.695 mmol/L);
3. low HDL cholesterol: <40 mg/dL (1.036 mmol/L) in men, and <50 mg/dL (1.295 mmol/L) in women;
4. high blood pressure: >130/85 mmHg; and
5. high-fasting glucose: >110 mg/dL (>6.1 mmol/L).

Metabolic syndrome may also be characterized by three or more of the following criteria: triglycerides >150 mg/dL, systolic blood pressure (BP)≧130 mmHg or diastolic BP≧85 mmHg, or on anti-hypertensive treatment, high-density lipoprotein cholesterol <40 mg/dL, fasting blood sugar (FBS)>110 mg/dL, and a body mass index (BMI)>28.8 k/m².

Metabolic syndrome may also be characterized by diabetes, impaired glucose tolerance, impaired fasting glucose, or insulin resistance plus two or more of the following abnormalities:
1. high blood pressure: ≧160/90 mmHg;
2. hyperlipidemia: triglyceride concentration ≧150 mg/dL (1.695 mmol/L) and/or HDL cholesterol <35 mg/dL (0.9 mmol/L) in men, and <39 mg/dL (1.0 mmol/L) in women;
3. central obesity: waist-to-hip ratio of >0.90 in men, and >0.85 in women and/or BMI >30 kg/m²; and
4. microalbuminuria: urinary albumin excretion rate ≧20 μg/min or an albumin-to-creatinine ratio ≧20 mg/g. Biomarkers include proteinuria, TGF-β, TNF-α and adiponectin.

Biomarkers include LDL, HDL and all the endothelial dysfunction markers.

The term “atrial fibrillation” (AF) refers to a type of irregular or racing heartbeat that may cause blood to collect in the heart and potentially form a clot which may travel to the brain and can cause a stroke.

The term “renal failure”, e.g., chronic renal failure; is characterized, e.g., by proteinuria and/or slight elevation of plasma creatinine concentration (106-177 mmol/L corresponding to 1.2-2.0 mg/dL).

The term “glomerulonephritis” refers to a condition which may be associated with the nephrotic syndrome, a high blood pressure and a decreased renal function, focal, segmental glomerulonephritis, minimal change nephropathy, Lupus nephritis, post-streptococal GN and IgA nephropathy.

The term “nephrotic syndrome” refers to a compilation of conditions including massive proteinuria, edema and central nervous system (CNS) irregularities. Biomarkers include urinary protein excretion.

The term “plaque stabilization” means rendering a plaque less dangerous by preventing, fibrous cap thinning/rupture, smooth muscle cell loss and inflammatory cell accumulation.

The term “renal fibrosis” refers to an abnormal accumulation of collagen and other extracellular matrix proteins, leading to loss of renal function. Biomarkers include collagen fragments and TGF-β in urine.

The term “end-stage renal disease” (ESRD) refers to loss of renal function to the extent that dialysis or renal replacement is needed. Biomarkers include glomerular filtration rate and creatinine clearance.

The term “polycystic kidney disease” (PKD) refers to a genetic disorder characterized by the growth of numerous cysts in the kidney. PKD cysts can slowly reduce much of the mass of kidneys reducing kidney function and leading to kidney failure. PKD may be classified as two major inherited forms of PKD which are autosomal dominant PKD and autosomal recessive PKD, while the non-inherited PKD may be called acquired cystic kidney disease. Biomarkers include reduction of renal cysts by non-invasive imaging.

The term “obesity” as used herein is a condition in which there is an excess of body fat. The operational definition of obesity is based on the Body Mass Index (BMI), which is calculated as body weight per height in meters squared (kg/m2). “Obesity” refers to a condition whereby an otherwise healthy subject has a Body Mass Index (BMI) greater than or equal to 30 kg/m2, or a condition whereby a subject with at least one co-morbidity has a BMI greater than or equal to 27 kg/m2. An “obese subject” is an otherwise healthy subject with a Body Mass Index (BMI) greater than or equal to 30 kg/m2 or a subject with at least one co-morbidity with a BMI greater than or equal to 27 kg/m2. A “subject at risk of obesity” is an otherwise healthy subject with a BMI of 25 kg/m2 to less than 30 kg/m2 or a subject with at least one co-morbidity with a BMI of 25 kg/m2 to less than 27 kg/m2. The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m2. In Asian countries, including Japan, an “obese subject” refers to a subject with at least one obesity-induced or obesity-related co-morbidity that requires weight reduction or that would be improved by weight reduction, with a BMI greater than or equal to 25 kg/m2. In Asia-Pacifc, a “subject at risk of obesity” is a subject with a BMI of greater than 23 kg/m2 to less than 25 kg/m2.

As used herein, the term “obesity” is meant to encompass all of the above definitions of obesity.

Obesity-induced or obesity-related co-morbidities include, but are not limited to, diabetes, non-insulin dependent diabetes mellitus—type 2, diabetes associated with obesity, impaired glucose tolerance, impaired fasting glucose, insulin resistance syndrome, dyslipidemia, hypertension, hypertension associated with obesity, hyperuricemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

The term “body fat reduction” means loss of a portion of body fat.

The term “muscle cells” refers to cells derived from the predominant cells of muscle tissue. Muscle cells may be freshly isolated from muscle tissue or established cell lines.

The term “weight loss” refers to loss of a portion of total body weight.

In the present description and in the claims, “appetency disorders” are understood as meaning disorders associated with a substance and especially abuse of a substance and/or dependency on a substance, disorders of food behaviors, especially those liable to cause excess weight, irrespective of its origin, for example: bulimia, appetency for sugars, non-insulin-dependent diabetes. Appetizing substances are therefore understood as meaning substances to be taken into the body and for which an appetite or craving for such consumption by any route of entry. Appetizing substances include, but are not limited to, foods, and their appetizing ingredients such as sugars, carbohydrates, or fats, as well as drinking alcohol or drugs of abuse or excess consumption. An “appetite’ may be directed toward such substances as foods, sugars, carbohydrates, fats, as well as ethanol or drugs of abuse or addiction or excess consumption (e.g., tobacco, CNS depressants, CNS stimulants). In one embodiment, the disorder is increased appetite associated with nicotine or tobacco withdrawal. Thus the term “appetency disorders” covers also treatment for reducing body weight or reducing body fat or reducing appetite for food or reducing food intake or consumption or causing hypophagia in mammals (e.g., humans, cats or dogs). The term “appetency disorders” can also cover a treatment to reduce appetite for food.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above. In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or; anxiolytic withdrawal with or without perceptual disturbances; sedative, hypnotic or anxiolytic withdrawal delirium; and withdrawal symptoms due to other substances. Thus the term “substance abuse disorders” covers also a treatment to suppress the increased appetite associated with nicotine or tobacco withdrawal, or the treatment of addiction to psychoactive substances such as narcotics, CNS stimulants, CNS depressants, and anxyiolytics. It will be appreciated that reference to treatment of nicotine withdrawal includes the treatment of symptoms associated with smoking cessation. Other “substance abuse disorders” include substance-induced anxiety disorder with onset during withdrawal; substance-induced mood disorder with onset during withdrawal; and substance-induced sleep disorder with onset during withdrawal.

The term “combination” comprising a therapeutic agent acting on the renin-angiotensin system (RAS) or a pharmaceutically acceptable salt thereof, and at least one CB1 antagonist, or a pharmaceutically acceptable salt thereof, means that the components can be administered together as a pharmaceutical composition or as part of the same, unitary dosage form. A combination also includes administering a therapeutic agent acting on the renin-angiotensin system (RAS) or a pharmaceutically acceptable salt thereof, and at least one CB1 antagonist, or a pharmaceutically acceptable salt thereof, each separately but as part of the same therapeutic regimen. The components, if administered separately, need not necessarily be administered at essentially the same time, although they can if so desired. Thus, a combination also refers, for example, administering a therapeutic agent acting on the renin-angiotensin system (RAS) or a pharmaceutically acceptable salt thereof, and at least one CB1 antagonist, or a pharmaceutically acceptable salt thereof, as separate dosages or dosage forms, but at the same time. A combination also includes separate administration at different times and in any order.

The renin inhibitors to which the present invention applies are any of those having renin inhibitory activity in vivo and, therefore, pharmaceutical utility, e.g., as therapeutic agents for the prevention of, delay the onset of and/or treatment of hypertension (whether for malignant, essential, reno-vascular, diabetic, isolated systolic, or other secondary type of hypertension), heart failure such as diastolic and congestive heart failure (acute and chronic), left ventricular dysfunction, endothelial dysfunction, diastolic dysfunction, hypertrophic cardiomyopathy, diabetic cardiac myopathy, supraventricular and ventricular arrhythmias, atrial fibrillation (AF), cardiac fibrosis, atrial flutter, detrimental vascular remodeling, plaque stabilization, myocardial infarction (MI) and its sequelae, atherosclerosis including coronary arterial disease (CAD), angina pectoris (whether unstable or stable), renal insufficiency (diabetic and non-diabetic), renal fibrosis, polycystic kidney disease (PKD), type 2 diabetes, metabolic syndrome, secondary aldosteronism, primary and secondary pulmonary hypertension, renal failure conditions such as nephrotic syndrome, diabetic nephropathy, glomerulonephritis, scleroderma, glomerular sclerosis, proteinuria of primary renal disease, renal vascular hypertension, diabetic retinopathy and end-stage renal disease (ESRD), the management of other vascular disorders such as migraine, peripheral vascular disease (PVD), Raynaud's disease, luminal hyperplasia, cognitive dysfunction (such as Alzheimer's), glaucoma and cerebrovascular disease such as embolic or thrombotic stroke.

In particular, the present invention relates to renin inhibitors disclosed in U.S. Pat. No. 5,559,111; No. 6,197,959 and No. 6,376,672, the entire contents of which are incorporated herein by reference.

Suitable renin inhibitors include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-proly 1-L-phenylalanyl-N-[2-hydroxy-5-methyl-1-(2-methylpropyl)-4-[[[2-methyl-1-[[(2-pyridinylmrthyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexy 1methyl)-2-hydroxy-3-(1-methylethoxy)-3-oxopropyl]-S-methyl-L-cysteineamide); and zankiren (chemical name: [1S-[1R*[R*(R*)],2S*,3R*]]-N-[1-(cyclohexylmethyl)-2,3-dihydroxy-5-m ethylhexyl]-alfa-[[2-[[(4-methyl-1-piperazinyl)sulfonyl]methyl]-1-oxo-3-phenylpropyl]-amino]-4-thiazolepropanamide), preferably, in each case, the hydrochloride salt thereof.

Preferred renin inhibitor of the present invention include RO 66-1132 and RO 66-1168 of formulae (I) and (II)

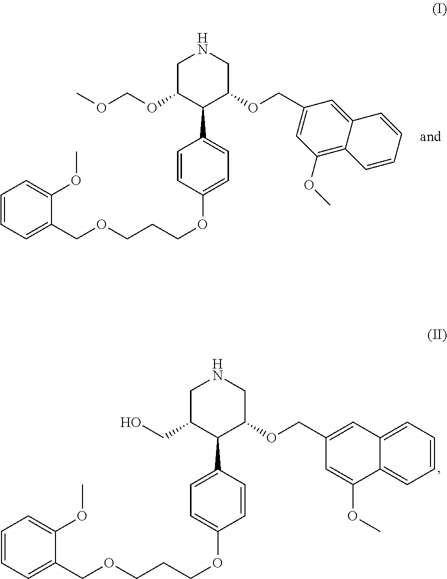

respectively, or a pharmaceutically acceptable salt thereof.

In particular, the present invention relates to a renin inhibitor which is a δ-amino-γ-hydroxy-ω-aryl-alkanoic acid amide derivative of the formula

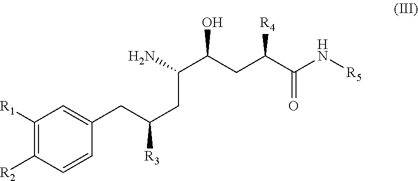

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆ alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (III) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₄alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxy-carbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are δ-amino-γ-hydroxy-ω-aryl-alkanoic acid amide derivatives of formula (III) having the formula

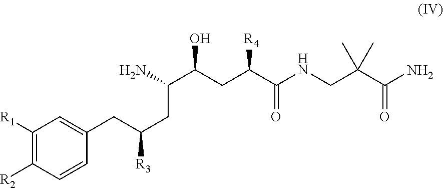

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren.

The term “aliskiren”, if not defined specifically, is to be understood both as the free base and as a salt thereof, especially a pharmaceutically acceptable salt thereof, most preferably a hemi-fumarate salt thereof.

Suitable angiotensin II receptor blockers which may be employed in the combination of the present invention include AT₁-receptor antagonists having differing structural features, preferred are those with the non-peptidic structures. For example, mention may be made of the compounds that are selected from the group consisting of valsartan (EP 443983), losartan (EP 253310), candesartan (EP 459136), eprosartan (EP 403159), irbesartan (EP 454511), olmesartan (EP 503785), tasosartan (EP 539086), telmisartan (EP 522314), the compound with the designation E-4177 of the formula

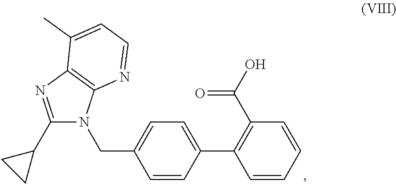

the compound with the designation SC-52458 of the following formula

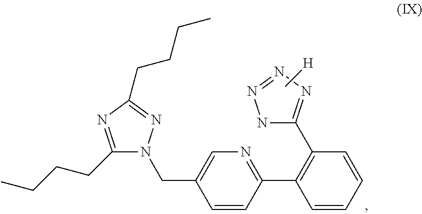

and the compound with the designation the compound ZD-8731 of the formula

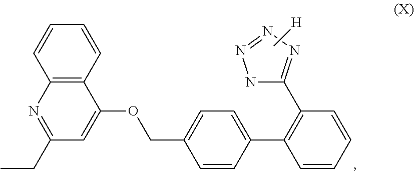

or, in each case, a pharmaceutically acceptable salt thereof.

Preferred AT₁-receptor antagonists are those agents that have reach the market, most preferred is valsartan, or a pharmaceutically acceptable salt thereof.

The class of ACE inhibitors which may be employed in the combination of the present invention comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

An antagonist of the CB1 cannabinoid receptor is a compound which binds to the receptor and lacks any substantial ability to activate the receptor itself. An antagonist can thereby prevent or reduce the functional activation or occupation of the receptor by an agonist such as anandamide when the agonist is present. In some embodiments, the antagonist has an IC₅₀ from about 1 μM to about 1 nM. In other embodiments, the antagonist has an IC₅₀ of from about 0.1 μM to 0.01 μM, 1.0 μM to 0.1 μM, or 0.01 μM to 1 nM. In some embodiments, the antagonist competes with the agonist for binding to a shared binding site on the receptor.

A first group of suitable cannabinoid CB1 receptor antagonists are pyrazole derivatives. Patent applications EP-A-576 357 and EP-A-658 546 describe exemplary pyrazole derivatives which have an affinity for the cannabinoid receptors. More particularly, patent application EP-A-656 354 discloses exemplary pyrazole derivatives and claims N-piperidino-5-(4-chlorophenyl)-1-(2,4-dichlorophenyl)-4-methylpyrazole-3-carboxamide, or SR 141716, and its pharmaceutically acceptable salts, which have a very good affinity for the central cannabinoid receptors. Additional exemplary CB1 receptor antagonists are disclosed in U.S. Pat. No. 5,596,106 which discloses both arylbenzo[b] thiophene and benzo[b]furan compounds to block or inhibit cannabinoid receptors in mammals. Preferably, such a cannabinoid antagonist is selective for the CB1 receptor and has an IC₅₀ for the CB1 receptor which is one-fourth or less than that of the CB2 receptor or, more preferably, is one-tenth or less than the IC₅₀ for the CB2 receptor, or even more preferably, an IC₅₀ with respect to the CB1 receptor which is one-hundredth that for the CB2 receptor. Each of the above references is incorporated by reference in its entirety.

Another representative example is Iodopravadoline (AM-630), which was introduced in 1995. AM-630 is a CB₁ receptor antagonist, but sometimes behaves as a weak partial agonist (Hosohata, K.; Quock, R. M.; Hosohata, Y.; Burkey, T. H.; Makriyannis, A.; Consroe, P.; Roeske, W. R.; Yamamura, H. I. Life Sc. 1997, 61, PL115). More recently, researchers from Eli Lilly described arylaroyl substituted benzofurans as selective CB₁ receptor antagonists (e.g. LY-320135) (Felder, C. C.; Joyce, K. E.; Briley, E. J.; Glass, M.; Mackie, K. P.; Fahey, K. J.; Cullinan, G. J.; Hunden, D. C.; Johnson, D. W.; Chaney, M. O.; Koppel, G. A.; Brownstein, M. J. Pharmacol. Exp. Ther. 1998, 284, 291). Recently, 3-alkyl-5,5′-diphenylimidazolidinediones were described as cannabinoid receptor ligands, which were indicated to be cannabinoid antagonists (Kanyonyo, M.; Govaerts, S. J.; Hermans, E.; Poupaert, J. H., Lambert, D. M. Biorg. Med. Chem. Lett. 1999, 9, 2233). Interestingly, many CB₁ receptor antagonists have been reported to behave as inverse agonists in vitro (Landsman, R. S.; Burkey, T. H.; Consroe, P.; Roeske, W. R.; Yamamura, H. I. Eur. J. Pharmacol. 1997, 334, R1). Recent reviews provide a nice overview of the current status in the cannabinoid research area (Mechoulam, R.; Hanus, L.; Fride, E. Prog. Med. Chem. 1998, 35, 199. Lambert, D. M. Curr. Med. Chem. 1999, 6, 635. Mechoulam, R.; Fride, E.; Di Marzo, V. Eur. J. Pharmacol. 1998, 359, 1). From the international patent application WO 01/70700 4,5-dihydro-1H-pyrazole compounds are known which exhibit potent and selective cannabis CB₁-receptor antagonistic activity.

Also useful are the cannabinoid CB1 receptor antagonist compounds of the formula

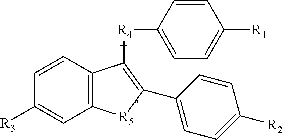

wherein the substituents R₁, R₂, R₃, R₄, and R₅ are defined as recited in U.S. Pat. No. 5,596,106 which is incorporated by reference in its entirety. Related reference U.S. Pat. No. 5,747,524 is also incorporated by reference in its entirety. This reference discloses additional exemplary aryl-benzo[b]thiophene and arylbenzo[b]furan derivatives for use according to the invention.

The cannabinoid antagonists of the following formula are also particularly useful according to the invention:

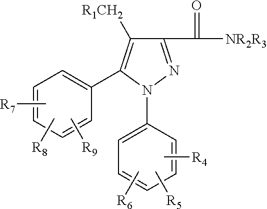

wherein R₁ is hydrogen, a fluorine, a hydroxyl, a (C₁-C₅)alkoxy, a (C₁-C₅)alkylthio, a hydroxy(C₁-C₅)alkoxy, a group —NR₁₀R₁₁, a cyano, a (C₁-C₅)alkylsulfonyl or a (C₁-C₅) alkylsulfinyl;
R₂ and R₃ are a (C₁-C₄)alkyl or, together with the nitrogen atom to which they are bonded, form a saturated or unsaturated 5- to 10-membered heterocyclic radical which is unsubstituted or monosubstituted or polysubstituted by a (C₁-C₃)alkyl or by a (C₁-C₃)alkoxy;
R₄, R₅, R₆, R₇, R₈ and R₉ are each independently hydrogen, a halogen or a trifluoromethyl, and if R₁ is a fluorine, R₄, R₅, R₆, R₇, R₈ and/or R₉ can also be a fluoromethyl, with the proviso that at least one of the substituents R₄ or R₇ is other than hydrogen; and
R₁₀ and R₁₁ are each independently hydrogen or a (C₁-C₅)alkyl, or R₁₀ and R₁₁, together with the nitrogen atom to which they are bonded, form a heterocyclic radical selected from pyrrolidin-1-yl, piperidin-1-yl, morpholin-4-yl and piperazin-1-yl, which is unsubstituted or substituted by a (C₁-C₄)alkyl,
   their salts and their solvates.

Other examples of selective CB₁ antagonistic compounds which are useful in the context of the present invention include (without being limited thereto):

1) Diarylpyrazole congeners disclosed by Sanofi as selective CB₁ receptor antagonists, e.g. as representative example the compounds SR-141716A, SR-147778, SR-140098 and rimonabant and related compounds described e.g. in EP 0969835 or EP 1150961(Central mediation of the cannabinoid cue: activity of a selective CB₁ antagonist, SR 141716A Perio A, Rinaldi-Carmona M, Maruani J Behavioural Pharmacology 1996, 7:1 (65-71)); WIN-54461 disclosed by Sanofi-Winthrop (Cannabinoid receptor ligands: Clinical and neuropharmacological considerations relevant to future drug discovery and development. Pertwee R G, Expert Opinion on Investigational Drugs 1996, 5:10 (1245-1253)). N-piperidino-5-(4-chlorophenyl)-1-(2,4-dichlorophenyl)-4-methylpyrazole-3-carboxamide (SR 141616—CAS number: 168273-06-1), its pharmaceutically acceptable salts and their solvates were described for the preparation of drugs useful in the treatment of appetency disorders. SR 141616, (pINN: rimonabant) is represented by the formula:

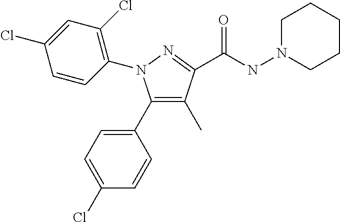

Rimonabant is specifically described in EP-B-656 354 or in an article from M. Rinaldi-Carmona et al. (FEBS Lett., 1994, 350, 240-244). EP1446384 A1 describes new polymorphs of rimonabant, formulation comprising rimonabant are described in WO2003082256, and the use of rimonabant in appetite disorders is described in WO99/00119.

2) Aminoalkylindoles having been disclosed as CB₁ receptor antagonists, e.g. as a representative example the compound Iodopravadoline (AM-630),

3) Aryl-aroyl substituted benzofurans described by Eli Lilly as selective CB₁ receptor antagonists, e.g. LY-320135 (Cannabinoid receptor ligands: Clinical and neuropharmacological considerations relevant to future drug discovery and development. Pertwee R G, Expert Opinion on Investigational Drugs 1996, 5:10 (1245-1253)),

4) Compounds described by Merck & Co, e.g. AM 251 and AM 281 (Conference: 31st Annual Meeting of the Society for Neuroscience, San Diego, USA, 10-15.11.2001), and substituted imidazolyl derivatives disclosed e.g. in U.S. 2003-114495 or WO 03/007887,

5) Azetidine derivatives described by Aventis Pharma e.g. in WO 02/28346 or EP 1328269,

6) CP-55940 from Pfizer Inc. (Comparison of the pharmacology and signal transduction of the human cannabinoid CB1 and CB2 receptors, Felder C C, Joyce K E, Briley E M, Mansouri J, Mackie K, Blond O, Lai Y, Ma A L, Mitchell R L, Molecular Pharmacology 1995, 48:3 (443)),

7) Diaryl-pyrazine-amide derivatives from Astra Zeneca described e.g. in the WO 03/051851,

8) ACPA and ACEA from Med. Coll. Wisconsin (Univ. Aberdeen), (“Effects of AM 251 & AM 281, cannabinoid CB1 antagonists, on palatable food intake in lewis rats” J. Pharmacol. Exp. Ther. 289, No 3, 1427-33, 1999),

9) Pyrazole derivatives described by the University of Conneticut e.g. in the WO 01/29007,

10) HU-210 (International Association for the Study of Pain—Ninth World Congress (Part II) Vienna, Austria, Dickenson A H, Carpenter K, Suzuki R, IDDB MEETING REPORT 1999, Aug. 22-27) and HU-243 (Cannabinoid receptor agonists and antagonists, Barth F, Current Opinion in Therapeutic Patents 1998, 8:3 (301-313)) from Yissum R&D Co Hebrew Univ. of Jerusalem,

11) O-823 from Organix Inc. (Drug development pipeline: O-585, O-823, O-689, O-1072, nonamines, Orgaix, Altropane Organix Inc, Company Communication 1999, Aug. 10; IDDb database) and O-2093 from Consiglio Nazionale delle Ricerche (“A structure/activity relationship study on arvanil, endocannabinoid and vanilloid hybrid.”, Marzo D V, Griffin G, Petrocellis L, Brandi I, Bisogno T, Journal of Pharmacology and Experimental Therapeutics 2002, 300:3 (984-991)),

12) 3-Alkyl-5,5′-diphenylimidazolidinediones which were described as cannabinoid receptor ligands,

13) CB₁ antagonistic compounds currently under development by Bayer AG (IDDb database: company communication 2002, Feb. 28).

14) CB1 receptor antagonists are pyrazole derivatives according to Formula (I) Of U.S. Pat. No. 6,028,084 which is incorporated by reference in its entirety.

15) U.S. Pat. No. 6,017,919 discloses another group of suitable cannabinoid receptor antagonists for use according to the invention. These antagonists

are of the following general formula:

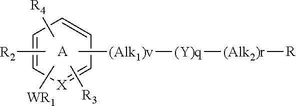

wherein the substituents are as defined in U.S. Pat. No. 6,017,919 which is incorporated herein by reference in its entirety.

16) The CB1 cannabinoid antagonist is a 4,5, dihydro-1H-pyrazole derivative having CB1-antagonist activity as taught in U.S. Pat. No. 5,747,524 and U.S. Patent Application No. 2001/0053788A1 published on Dec. 20, 2001.

17) The CB1 receptor antagonist is a 4,5,dihydro-1H-pyrazole derivative having CB1-antagonistic activity as taught in U.S. Patent Application No. 2001/0053788A1 and particularly disclosed by formula (I) therein. U.S. Patent Application No. 2001/0053788A1 published on Dec. 20, 2001 and is incorporated by reference in its entirety.

18) The CB1 receptor antagonists described in WO2005049615 especially the compounds of example 1 to 8.

19) The CB1 receptor antagonists described in WO2005047285 especially the compounds of example 1 to 99.

20) The CB1 receptor antagonist (4R)-3-(4-chlorophenyl)-4,5-dihydro-N-methyl-4-phenyl-N′-[[4-(trifluoromethyl)phenyl]sulfonyl]-1H-pyrazole-1-carboximidamide (SLV 326-34^{th} Neuroscience, Abs 1009.4, October 2004)

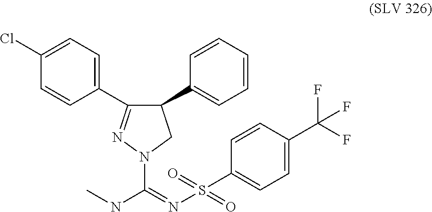

developed by the company Solvay (WO0170700 A1).

Solvay CB1 receptor antagonists are described in the examples of the patent applications WO2005040130 A1, WO2005028456 A1, WO2005020988 A1, WO2004026301 A1, WO2003078413 A1, WO2003027076 A2, WO2003026648 A1, WO2003026647 A1, WO2002076949 A1, WO0170700 A1.

Particularly preferred are CB1 receptor antagonists selected from the group consisting of rimonabant, AM-630, AM251, AM281, LY-320135, HU-210, HU-243, O-823, O-2093, SLV 326 and SR147778, preferably rimonabant, AM251 or SR147778, more preferably rimonabant, or, in each case, a pharmaceutically acceptable salt thereof.

Any of the substances for the respective class as disclosed in the above mentioned patent documents or scientific publications, hereby included by reference, are considered potentially useful to be used in carrying out the present invention.

In each case in particular in the compound claims and the final products of the working examples, the subject matter of the final products, the pharmaceutical preparations and the claims are hereby incorporated into the present application by reference to these publications.

Preferred are combinations, such as combined preparations or pharmaceutical compositions, respectively, comprising a renin inhibitor, e.g., aliskiren, especially in the form of the hemi-fumarate salt thereof and as the second active agent an active agent selected from the group consisting of Rimonabant, AM-630, AM251, AM281, LY-320135, HU-210, HU-243, O-823, O-2093, SLV 326 or in any case a pharmaceutically accepted salt thereof.

Preferred are combinations, such as combined preparations or pharmaceutical compositions, respectively, comprising an angiotensin II receptor blocker (ARB), e.g. valsartan or a pharmaceutically acceptable salt thereof and as the second active agent an active agent selected from the group consisting of Rimonabant, AM-630, AM251, AM281, LY-320135, HU-210, HU-243, O-823, O-2093, SLV 326 or in any case a pharmaceutically accepted salt thereof.

Preferred are combinations, such as combined preparations or pharmaceutical compositions, respectively, comprising an angiotensin converting enzyme (ACE) inhibitor, e.g. benazepril or a pharmaceutically acceptable salt thereof and as the second active agent an active agent selected from the group consisting of Rimonabant, AM-630, AM251, AM281, LY-320135, HU-210, HU-243, O-823, O-2093, SLV 326 or in any case a pharmaceutically accepted salt thereof.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts.

Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

All the more surprising is the experimental finding that the combined administration of a therapeutic agent acting on the renin-angiotensin system (RAS) or a pharmaceutically acceptable salt thereof and at least one CB1 antagonist or a salt thereof, results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of a therapeutic agent acting on the renin-angiotensin system (RAS) with at least one CB1 antagonist results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects on diseases and disorders that may be modulated by action on the renin-angiotensin system (RAS), obesity, appetency disorders or substance abuse disorders, in particular obesity, or appetency disorders, for a number of combinations as described herein.

Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the combination of the active agents used according to the present invention can be demonstrated e.g. by using corresponding pharmacological models known in the pertinent art.

Accordingly, the combination according to the present invention may be used, e.g., for the prevention, delay of progression or treatment of diseases and disorders that may be modulated by action on the renin-angiotensin system (RAS) and/or appetency disorders or nicotinic addiction.

Thus in a further aspect the present invention concerns the use of the above combination for the manufacture of a medicament for the prevention, delay of progression or treatment of diseases and disorders that may be modulated by action on the renin-angiotensin system (RAS), obesity, appetency disorders or substance abuse disorders.

The invention furthermore relates to a method for the prevention of, delay of progression of, treatment of diseases and disorders that may be modulated by action on the renin-angiotensin system (RAS), obesity, appetency disorders or substance abuse disorders, comprising administering to a warm-blooded animal, including man, in need thereof an effective amount of the above combination.

Methods or uses as described above, wherein the disease or condition is selected from obesity, appetency disorders and substance abuse disorders or for body fat reduction.

More preferably, the disease or condition is appetency disorders or substance abuse disorders, or for body fat reduction.

Most preferably, the disease or condition is selected from obesity or appetency disorders.

In one further embodiment, the herein described methods, uses and compositions are used to suppress the increased appetite associated with nicotine or tobacco withdrawal.

In one further embodiment, the herein described methods, uses and compositions are used for body fat reduction.

Preferred combinations for the described uses or methods are described herein.

Preferably, the jointly therapeutically effective amounts of the active agents according to the combination of the present invention can be administered simultaneously or sequentially in any order, e.g. separately (combined pharmaceutical preparation) or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of therapeutic agent acting on the renin-angiotensin system (RAS) and a CB1 antagonist according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes (e.g. less appetite, less gain of weight or less cardiovascular side effects).

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of hypertensive patients, e.g. reducing the risk of negative cardiovascular events, reducing risk of side effects, controlling increase of weight (in diabetic patients) or in patients suffering from an altered gastrointestinal motility, sensitivity and/or secretion disorder(s).

In view of reduced dose of therapeutic agent acting on the renin-angiotensin system (RAS) or CB1 antagonist, used according to the present invention, there is a considerable safety profile of the combination making it suitable for first line therapy.

The pharmaceutical composition according to the present invention as described herein before and hereinafter may be used for simultaneous use or sequential use in any order, for separate use or as a fixed combination.

Method or use as described above, wherein therapeutic agent acting on the renin-angiotensin system (RAS) and the CB1 antagonist are administered in the form of a combination of the present invention such as a fixed combination or combined preparation or kit of part.

“kit-of-parts”, combination, method or use as described herein, wherein the therapeutic agent acting on the renin-angiotensin system (RAS) is aliskiren or and wherein the CB1 antagonist is preferably selected from the group consisting of Rimonabant, AM-630, AM251, AM281, LY-320135, HU-210, HU-243, O-823, O-2093, SLV 326, or in each case, a pharmaceutically acceptable salt thereof.

“kit-of-parts”, combination, method or use as described above, wherein the therapeutic agent acting on the renin-angiotensin system (RAS) is aliskiren and wherein the CB1 antagonist is Rimonabant, or in each case, a pharmaceutically acceptable salt thereof.

“kit-of-parts”, combination, method or use as described herein, wherein the therapeutic agent acting on the renin-angiotensin system (RAS) is valsartan or and wherein the CB1 antagonist is preferably selected from the group consisting of Rimonabant, AM-630, AM251, AM281, LY-320135, HU-210, HU-243, O-823, O-2093, SLV 326, or in each case, a pharmaceutically acceptable salt thereof.

“kit-of-parts”, combination, method or use as described above, wherein the therapeutic agent acting on the renin-angiotensin system (RAS) is valsartan and wherein the CB1 antagonist is Rimonabant, or in each case, a pharmaceutically acceptable salt thereof.

“kit-of-parts”, combination, method or use as described herein, wherein the therapeutic agent acting on the renin-angiotensin system (RAS) is benazepril or and wherein the CB1 antagonist is preferably selected from the group consisting of Rimonabant, AM-630, AM251, AM281, LY-320135, HU-210, HU-243, O-823, O-2093, SLV 326, or in each case, a pharmaceutically acceptable salt thereof.

“kit-of-parts”, combination, method or use as described above, wherein the therapeutic agent acting on the renin-angiotensin system (RAS) is benazepril and wherein the CB1 antagonist is Rimonabant, or in each case, a pharmaceutically acceptable salt thereof.

According the invention, when the therapeutic agent acting on the renin-angiotensin system (RAS) and the CB1 antagonist are administered together, such administration can be sequential in time or simultaneous with, the simultaneous method being generally preferred. For sequential administration, the therapeutic agent acting on the renin-angiotensin system (RAS) and the CB1 antagonist can be administered in any order. It is generally preferred that such administration be oral. It is especially preferred that the administration be oral and simultaneous. However, if the subject being treated is unable to swallow, or oral absorption is otherwise impaired or undesirable, parenteral or transdermal administration will be appropriate. When the therapeutic agent acting on the renin-angiotensin system (RAS) and the CB1 antagonist are administered sequentially, the administration of each can be by the same method or by different methods.

A further aspect of the present invention is a kit for the prevention of, delay of progression of, treatment of a disease or condition according to the present invention comprising

(a) an amount of the therapeutic agent acting on the renin-angiotensin system (RAS) or a pharmaceutically acceptable salt thereof in a first unit dosage form;
(b) an amount of at least one CB1 antagonist or, in each case, where appropriate, a pharmaceutically acceptable salt thereof in a second etc. unit dosage form; and
(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of the therapeutic agent acting on the renin-angiotensin system (RAS) or a pharmaceutically acceptable salt thereof in a first unit dosage form;
(b) an amount of at least one CB1 antagonist or, in each case, where appropriate, a pharmaceutically acceptable salt thereof,
in the form of two or three or more separate units of the components (a) to (b), especially for the prevention of, delay of progression of, treatment of a disease or condition according to the present invention.

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (a) or (b)), together with instructions for its simultaneous, separate or sequential use, or any combination thereof, in the delay of progression or treatment of the diseases as mentioned herein.

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubilizing or lyophilizing processes. Thus, pharmaceutical preparations for oral use can be obtained by combining the active compound(s) with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical composition according to the present invention as described hereinbefore may be used for simultaneous use or sequential use in any order, for separate use or as a fixed combination.

Thus according to a further embodiment, the therapeutic agent acting on the renin-angiotensin system (RAS) is administered with a CB1 antagonist, preferably in the form of a fixed pharmaceutical composition comprising a pharmaceutically acceptable carrier, vehicle or diluent. Accordingly, the therapeutic agent acting on the renin-angiotensin system (RAS) of this invention, can be administered with a CB1 antagonist as a fixed combination, in any conventional oral, parenteral or transdermal dosage form.

The doses of the therapeutic agent acting on the renin-angiotensin system (RAS) to be administered to warm-blooded animals, for example human beings, of, for example, approximately 70 kg body weight, will be generally dependent upon the health of the subject being treated, the extent of treatment desired, the nature and kind of concurrent therapy, if any, and the frequency of treatment and nature of the effect desired. In general, the dosage of the agent is generally in the range of from about 0.001 to about 50 mg/kg body weight of the subject per day, preferably from about 0.1 to about mg/kg body weight of the subject per day, administered as a single or divided dose. However, some variability in the general dosage range may also be required depending upon the age, weight, and species of the patient, the intended route of administration, and the progress and degree of severity of the disease or condition being treated.

Daily dosages of the therapeutic agent acting on the renin-angiotensin system (RAS) required in practicing the method of the present invention will vary depending upon, for example the mode of administration and the severity of the condition to be treated. An indicated daily dose is in the range of from about 1 to about 500 mg, e.g. from 1 to 100 mg of active agent for oral use, conveniently administered once or in divided dosages. Normally, in the case of oral administration, an approximate daily dose of from about 1 mg to about 360 mg is to be estimated, e.g., for a patient of approximately 75 kg in weight.

For example, the doses of aliskiren to be administered to warm-blooded animals, including man, of approximately 75 kg body weight, especially the doses effective for the inhibition of renin activity, e.g., in lowering blood pressure, are from about 3 mg to about 3 g, preferably from about 10 mg to about 1 g, e.g., from 5 to 500 mg, preferably 20 to 200 mg/person/day, divided preferably into 1 to 4 single doses which may, e.g., be of the same size. Usually, children receive about half of the adult dose. The dose necessary for each individual can be monitored, e.g., by measuring the serum concentration of the active ingredient, and adjusted to an optimum level. Single doses comprise, e.g., 75 mg, 150 mg or 300 mg per adult patient.

Angiotensin II receptor blockers, e.g., valsartan, are supplied in the form of a suitable dosage unit form, e.g., a capsule or tablet, and comprising a therapeutically effective amount of an angiotensin II receptor blocker, e.g., from about 20 to about 320 mg of valsartan, which may be applied to patients. The application of the active ingredient may occur up to three times a day, starting, e.g., with a daily dose of 20 mg or 40 mg of an angiotensin II receptor blocker, e.g., valsartan, increasing via 80 mg daily and further to 160 mg daily, and finally up to 320 mg daily. Preferably, an angiotensin II receptor blocker, e.g., valsartan is applied once a day or twice a day with a dose of 80 mg or 160 mg, respectively, each. Single doses comprise, e.g., 40 mg, 80 mg or 160 mg per adult patient. Corresponding doses may be taken, e.g., in the morning, at mid-day or in the evening.

In case of ACE inhibitors, preferred dosage unit forms of ACE inhibitors are, for example, tablets or capsules comprising e.g. from 3 to 40 mg, preferably from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is t.i.d. administration.

The dosage of CB1 antagonist administered will also be generally dependent upon the health of the subject being treated, the extent of treatment desired, the nature and kind of concurrent therapy, if any, and the frequency of treatment and nature of the effect desired. In general, the dosage of the agent is generally in the range of from about 0.001 to about 50 mg/kg body weight of the subject per day, preferably from about 0.1 to about 10 mg/kg body weight of the subject per day, administered as a single or divided dose. However, some variability in the general dosage range may also be required depending upon the age, weight, and species of the patient, the intended route of administration, and the progress and degree of severity of the disease or condition being treated.

Daily dosages of the agent interacting with a CB1 antagonist required in practicing the method of the present invention will vary depending upon, for example the mode of administration and the severity of the condition to be treated. An indicated daily dose is in the range of from about 1 to about 500 mg, e.g. from 1 to 100 mg of active agent for oral use, conveniently administered once or in divided dosages.

The preferred herein mentioned CB1 antagonists will be supplied in the form of suitable dosage unit form, for example, a capsule or tablet, and comprising a therapeutically effective amount, e.g. from about 2 to about 200 mg, as already described herein and in the prior art. The application of the active ingredient may occur up to three times a day, preferably one or two times a day. The same preferred dosage are selected for the fixed combinations.

Daily rimonabant dosages required in practicing the method of the present invention will vary depending upon, for example the mode of administration and the severity of the condition to be treated. An indicated daily dose is in the range of from about 1 to about 100 mg, e.g. from 5 to 40 mg or from 5 to 20 mg, of active agent for oral use, conveniently administered once or in divided dosages.

Corresponding doses may be taken, for example, in the morning, at mid-day or in the evening.

In a preferred aspect, the invention concerns a “kit-of-parts”, combination, use or a method as described herein, comprising or wherein the daily administration is;
i) of from 50 to 500 mg of aliskiren, and
ii) of from 5 and 50 mg or between 5 and 20 mg of rimonabant,
   or in any case, a pharmaceutically acceptable salt thereof.

In a preferred aspect, the invention concerns a “kit-of-parts”, combination or use or a method as described herein, comprising or wherein the daily administration is;
i) 75, 150 or 300 mg of aliskiren, and
ii) 5, 10 or 20 mg of rimonabant,
   or in any case, a pharmaceutically acceptable salt thereof.

Preferably, in case of free combinations, preferred are those dosages for launched products that have been approved and that have been marketed.

Especially preferred are low dose combinations.

To further illustrate the invention, but not by way of limitation, the following examples are provided.

A) Bioassay Methods for Assessing the Effects of Compounds and Combination Therapies on Appetite(s), Body Fat Reduction, Body Weight, and Lipid Metabolism.

The dose(s) administered to the animal are sufficient to determine if the compounds or combination therapy has a desired effect, for example, an appetite, body weight, body fat, and/or fatty acid oxidation over time. Such dose(s) can be determined according to the efficacy of the particular candidate compound(s) employed and the condition of the animal, as well as the body weight or surface area of the animal. The size of the dose(s) also will be determined by the existence, nature, and extent of any adverse side effects that accompany the administration of a candidate compound or combination; the LD50 of the candidate compound or combination; and the side-effects of the candidate compound or combination at various concentrations. Depending upon the compound or combination and the above factors, for instance, the initial test dosage(s) may range, for example, from 0.1-50 mg per kg, preferably 1-25 mg per kg, most preferably 1-20 mg per kg body weight for each of the compound or combination. The determination of dose response relationships is well known to one of ordinary skill in the art.

Test animals subjects can be, for example, obese or normal mammals (e.g., humans, primates, guinea pigs, rats, mice, or rabbits). Suitable rats include, but are not limited to, Zucker rats. Suitable mice include, but are not limited to, for example, ALS/LtJ, C3. SW-H-2b/SnJ, (NON/LtJ x NZO/HIJ)F1, NZO/H1J, ALR/LtJ, NON/LtJ, KK.Cg-AALR/LtJ, NON/LtJ, KK.Cg-Ay/J, B6.HRS(BKS)-Cpefat/+, B6.129P2-Gcktm/Efr, B6.V-Lepob, BKS.Cg-m+/+Leprdb, and C57BL/6J with Diet Induced Obesity.

### A. Assessing Effects on Appetite, Including Food Consumption.

The effect of the candidate compounds and combinations i.e. therapeutic agents acting on the renin-angiotensin system (RAS) (aliskiren) and CB1 antagonists (rimonabant) or combination of such compounds on an appetite for appetizing substance (e.g., sugar, ethanol, a psychoactive substance such as nicotine, narcotics, opiates, CNS stimulants or depressants, anxyiolytic) can be assessed, for instance, by monitoring the consumption of the substance by test subjects (e.g., measuring the amount (e.g., by volume or weight) Consumed or used or not consumed and not used, use of consumption diaries) Or tissue levels (e.g., blood, plasma) Or excretion levels (e.g., urine, feces levels) of the appetitive substance or its metabolites or by monitoring behaviors seeking the appetitive substance. The effect of the compounds and combinations on appetite can also be assessed by subjective means including questionnaires as to appetite or cravings levels by human subjects. The techniques for these assessments are well known to those of ordinary skill in the art. The studies may be acute, subacute, chronic, or subchronic with respect to the duration of administration and or follow-up of the effects of the administration. See also U.S. Pat. No. 6,344,474.

The effect of the candidate compounds and combinations i.e. therapeutic agents acting on the renin-angiotensin system (RAS) (aliskiren) and CB1 antagonists (rimonabant) or combination of such compounds on the appetite for food or in inducing hypophagia or reduced food intake can be directly assessed, for instance, by monitoring the food consumption of the test subjects (e.g., measuring the amount eaten or not eaten by a subject in terms of food weight or caloric content). The effect on food consumption can be indirectly measured by monitoring body weight. The effect of the compounds on appetite can also be assessed by food consumption diaries, or subjective means including questionnaires as to appetite or food cravings levels by human subjects. The techniques for these assessments are well known to those of ordinary skill in the art. The studies may be acute, subacute, chronic, or subchronic with respect to the duration of administration and or follow-up of the effects of the administration.

### B) Assessing Effects on Body Fat Reduction.

Effects on body fat can be identified in vivo using animal bioassay techniques well known to those of ordinary skill in the art. Body fat reduction is typically determined by direct measurements of the change in body fat or by loss of body weight. Body fat and/or body weight of the animals is determined before, during, and after the administration of the candidate compounds or combinations. Test compounds (therapeutic agents acting on the renin-angiotensin system (aliskiren) and CB1 antagonists (rimonabant)) or combinations thereof and appropriate vehicle or caloric controls can be administered by any of a number of routes (e.g., the oral route, a parenteral route) to experimental subjects and the weight of the subjects can be monitored over the course of therapy. The experimental subjects can be humans as well as surrogate test animals (e.g., rats, mice).

Changes in body fat are measured by any means known in the art such as, for example, fat fold measurements with calipers, bioelectrical impedance, hydrostatic weighing, or dual x-ray absorbiometry. Preferably animals demonstrate at least 2%, 5%, 8%, or 10% loss of body fat. Changes in body weight can be measured by any means known in the art such as, for example, on a portable scale, on a digital scale, on a balance scale, on a floor scale, or a table scale. Preferably animals demonstrate at least 2%, 5%, 10%, or 15% loss of body weight. Body weight reduction is measured before administration of the candidate compound or combination and at regular intervals during and after treatment. Preferably, body weight is measured every 5 days, more preferably every 4 days, even more preferably every 3 days, yet more preferably every 2 days, most preferably every day.

For instance, the effect of the candidate compounds and combinations on total body fat can be determined by taking direct measurements of the rat's body fat using skin fold calipers. Skin on the subjects' backs, abdomen, chest, front and rear legs can be pinched with calipers to obtain measurements before administration of the test compound and at daily or longer intervals (e.g., every 48 hours) during and after administration of candidate compounds and combinations. Differences in measurements in one or more of the “pinched” sites reflect the change in the rat's total body fat. The animal may selected from any test species, including but not limited to, mammals, the mouse, a rat, a guinea pig, or a rabbit. The animal may also be an ob/ob mouse, a db/db mouse, or a Zucker rat or other animal model for a weight-associated disease. Clinical studies in humans may also be conducted. In humans, body density measurements or estimates of percent body fat may also be used to assess body fat reduction.

### C) Assessing Effects on Lipid Metabolism.

The candidate compounds and combinations i.e therapeutic agents acting on the renin-angiotensin system (RAS) (aliskiren) and CB1 antagonists (rimonabant) or combinations of such compounds can also be assayed for their effect on fatty acid metabolism. The effect of the candidate compounds and combinations on fatty acid metabolism can be measured by measurements of fatty acid oxidation in primary cultures of liver cells as taught for instance in U.S. patent application Ser. No. 10/112,509 filed on Mar. 27, 2002 and assigned to the same assignee as the present application and incorporated by reference.

Changes in fatty acid metabolism can be measured, for instance, by looking at fatty acid oxidation in cells from major fat burning tissues such as, for example, liver (Beynen, et al., Diabetes, 28:828 (1979)), muscle (Chiasson Lab. Anat. of Rat (1980)), heart (Flink, et al., J. Biol. Chem., 267: 9917 (1992)), and adipocytes (Rodbell, J. Biol. Chem., 239: 375 (1964)), Cells may be from primary cultures or from cell lines. Cells may be prepared for primary cultures by any means known in the art including, for example, enzymatic digestion and dissection. Suitable cell lines are known to those in the art. Suitable hepatocyte lines are, for example, Fao, MH1C1, H-4-II-E, H4TG, H4-II-E-C3, McA-RH7777, McA-RH8994, N1-S1 Fudr, N1-S1, ARL-6, Hepa 1-6, Hepa-1c1c7, BpRc1, tao BpRc1, NCTC clone 1469, PLC/PRF/5, Hep 3B2.1-7 [Hep 3B], Hep G2 [HepG2], SK-HEP-1, WCH-17. Suitable skeletal muscle cell lines are, for example, L6, L8, C8, NOR-10, BLO-11, BC3H1, G-7, G-8, C2C12, P19, So18, SJRH30 [RMS13], QM7. Suitable cardiac cell lines are, for example, H9c2(2-1), P19, CCD-32Lu, CCD-32Sk, Girardi, FBHE. Suitable adipocyte lines are, for example, NCTC clone 929 [derivative of Strain L; L-929; L cell], NCTC 2071, L-M, L-M(TK-) [LMTK-; LM(tk-)], A9 (APRT and HPRT negative derivative of Strain L), NCTC clone 2472, NCTC clone 2555, 3T3-L1, J26, J27-neo, J27-B7, MTKP 97-12 pMp97B [TKMp97-12], L-NGC-5HT2, Ltk-11, L-alpha-1b, L-alpha-2A, L-alpha-2C, B82.

The rate of fatty acid oxidation may be measured by 14C-oleate oxidation to ketone bodies (Guzmán and Geelen Biochem. J. 287:487 (1982)) and/or 14C-oleate oxidation to CO₂ (Fruebis, PNAS, 98:2005 (2001); Blazquez, et al., J. Neurochem, 71: 1597 (1998)). Lypolysis may be measured by fatty acid or glycerol release by using appropriate labeled precursors or spectrophotometric assays (Serradeil-Le Gal, FEBS Lett, 475: 150 (2000)). For analysis of 14C-oleate oxidation to ketone bodies, freshly isolated cells or cultured cell lines can be incubated with 14C-oleic acid for an appropriate time, such as, for example, 30, 60, 90, 120, or 180 minutes. The amount of 14C radioactivity in the incubation medium can be measured to determine their rate of oleate oxidation. Oleate oxidation can be expressed as nmol oleate produced in x minutes per g cells. For analysis of lypolysis/glycerol release, freshly isolated cells or cultured cells lines can be washed then incubated for an appropriate time. The amount of glycerol released into the incubation media can provide an index for lypolysis.

### D) Cannabinoid Receptor Activity Screening.

A variety of means may be used to screen cannabinoid CB1 receptor activity in order to identify the compounds according to the invention. A variety of such methods are taught in U.S. Pat. No. 5,747,524 and U.S. Pat. No. 6,017,919.

To evaluate the antihypertensive activity of the combination according to the invention, for example, the methodology as described by Lovenberg W: Animal models for hypertension research. Prog. Clin. Biol. Res. 1987, 229, 225-240 may be applied. For the evaluation that the combination according to the present invention may be used for the treatment of congestive heart failure, for example, the methods as disclosed by Smith H J, Nuttall A: Experimental models of heart failure. Cardiovasc Res 1985, 19, 181-186 may be applied. Molecular approaches such as transgenic methods are also described, for example by Luft et al.: Hypertension-induced end-organ damage, “A new transgemic approach for an old problem”, Hypertension 1999, 33, 212-218.

The person skilled in the pertinent art is fully enabled to select a relevant test model to prove the efficacy of a combination of the present invention in the hereinbefore and hereinafter indicated therapeutic indications. Human renin inhibitors, for example aliskiren, typically have a high species specificity, most notably for human renin. Unexpectedly, aliskiren retains reasonable potency against dog and mouse renin but is 1-2 orders of magnitude weaker as a rat renin inhibitor. Consequently, rat models are not the preferred species but can be used if either higher doses of aliskiren (or another renin inhibitor) are used or if an animal species is used in which the RAS is particularly activated or artificially ‘over-activated’. A relevant and useful model for evaluating the effects of a combination of a renin inhibitor with a drug to treat obesity is the dog fed a high fat diet to induce obesity. These animals are hypertensive and overweight and have a generalized disturbance of metabolic parameters (Hall J E, Brands M W, Dixon W N, Smith M J Jr. Obesity-induced hypertension. Renal function and systemic hemodynamics. Hypertension 22:292-299, 1993). Some mouse models such as the Agouti Yellow Obese Mouse (Correia M L G, Haynes W G, Rahmouni K, Morgan D A, Sivitz W I, Mark A L. The concept of selective leptin resistance. Diabetes 51:439-442, 2002.

) or the transgenic mouse with reduced brown fat (Cittadini A, Mantzoros C S, Hampton T G, Travers K E, Katz S E, Morgan J P, Flier J S, Douglas P S. Cardiovascular abnormalities in transgenic mice with reduced brown fat. Circulation 100:2177-2183, 1999) can serve as useful models of human obesity with superimposed hypertension. A less preferred, but still useful animal model is the stroke prone spontaneously hypertensive rat (Izumo strain) crossed with Zucker fatty (ZF)(fa/fa) rats to yield the SHRSP fatty (fa/fa) rat (Hiraoka-Yamamoto et al., 2004). Additionally, Zucker rats (OZR or fa/fa) or its diabetic relative, the Zucker Diabetic Fatty Rat (ZDF) may also be utilized. These animal models are characterized by hypertension, obesity, insulin resistance/glucose intolerance (OZR) or diabetes (ZDF) and hyperlipidemia (Toblli J E, DeRosa G, Rivas C, Cao G, Piorno P, Pagano P, Forcada P. Cardiovascular protective role of a low-dose antihypertensive combination in obese Zucker rats. J Hypertens 21:611-620, 2003; van Zwieten P A. Diabetes and hypertension: experimental models for pharmacological studies. Clin Exp Hypertens 21:1-6, 1999, Mizuno M, Sada T, Kato M, Koike H. Renoprotective effects of blocakde of angiotensin II AT1 receptors in an animal model of type 2 diabetes. Hypertens Res 25(2):271-278, 2002). Although the effects of the combination can be evaluated in OZR of any age, the metabolic parameters will vary according to the age of the animal. Older animals may present with more substantial structural and functional changes than young rats due to the long-standing metabolic disturbance and the impact of these changes on the overall disease process. Therefore, results from laboratory to laboratory may vary due to the age at which the animals are used for study. Animals are typically 10-20 weeks of age when experiments are initiated. Typically, measurement of a wide variety of metabolic and functional parameters, including plasma lipids, plasma glucose, glucose tolerance, plasma insulin, body weight, and blood pressure, are made. Other more specific measurements may also be performed to assess endothelial function, oxidative stress, organ weight determinations, assessment of cardiac mass, cardiac and renal function and morphometric analyses. Refer to Zhou M S, Jaimes E A, Raij L. Atorvastatin prevents end-organ injury in salt-sensitive hypertension. Hypertens 44:186-190, 2004 for a description of some of these measurements. Blood pressure is also monitored chronically and with greater consistency using radiotelemetry as described below.

Radiotransmitters are implanted into either rats of at least 7 weeks of age or with body weight greater than 200 grams. In mice, body weight should exceed 20 grams at the time of telemetry implantation. Drug treatment can be initiated at any time following a two week recovery period from surgery. Drugs are administered once daily by oral gavage but may be given by other routes (eg., intra-peritoneal, intra-venous, or subcutaneous). Rats or mice are randomized to receive one of the various treatments, including a vehicle control. Drugs are administered by oral gavage, once daily in the morning for several weeks to 2 or 3 months. In special cases, drugs may be administered in the evening or multiple times per day. Also, for some studies in which the effects of feeding or behavior is to be noted, rats or mice can be placed on a reverse lighting schedule to induce a diurnal shift in eating and drinking patterns. Blood pressure (mean, systolic, and diastolic) and heart rate are continuously monitored, 24 hours per day for the full duration of the study using radiotelemetric procedures. All values depict 24 hour average responses for each animal but data summarization may also be performed using other time intervals, for example, hourly averaging. Body weights were recorded at weekly intervals or in some studies, may be monitored daily. Upon completion of the study, all rats or mice are sacrificed and hearts removed, sectioned and weighed. Cardiac mass was determined as the left ventricular weight to body weight ratio for each animal within a treatment group. Other tissues, including but not restricted to the kidney, may be removed at sacrifice for determination of biochemical markers, to assess the extent of tissue damage (histology, immunohistochemistry, etc), and for gene expression profiling. Blood sampling for measurement of glucose, insulin, lipids or other biochemical markers of metabolic function can be performed at various time points but is specifically limited (blood volume and frequency) depending upon the species. Thus, in a dog model, more frequent blood sampling and larger volumes are possible and consequently, a more extensive biochemical marker analysis can be performed.

### Determining the Combination Therapy Dosages.

Preferred dosages of the CB1 antagonist and the therapeutic agent acting on the renin-angiotensin system (RAS) to be used in a combination therapy can be determined experimentally by first conducting separate dose response studies for the CB1 antagonist the therapeutic agent acting on the renin-angiotensin system (RAS) to be used. Methods of performing such dose response studies in a test species or the species of the intended subject (e.g., a human) are well known to one of ordinary skill in the art. The endpoint of the study is preferably selected according to the effect or endpoint of interest (e.g., appetite reduction, weight loss, body fat reduction, changes in lipid metabolism, changed food seeking behavior) Or the dose response of the underlying mechanism of action (e.g., receptor activation or antagonism). Alternatively, the established dose response relationships may be used if an agent is already well-characterized as to dose response. Preferred bioassay methods include those described above and those presented in the Examples.

### EXAMPLE 1

Composition of aliskiren 150 mg (free base) uncoated tablets in mg/unit.

| | Roller | | | |
|---|---|---|---|---|
| | compacted | Dosage | Dosage | Dosage |
| Component | tablet | form 1 | form 2 | form 3 |
| Aliskiren hemi-fumarate | 165.750 | 165.750 | 165.750 | 165.750 |
| Microcrystalline cellulose | 220.650 | 84.750 | 72.250 | 107.250 |
| Polyvinylpyrrolidon K 30 | — | — | 12.000 | 12.000 |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

Composition of aliskiren 150 mg (free base) uncoated tablets in % by weight.

| | Roller | | | |
|---|---|---|---|---|
| | compacted | Dosage | Dosage | Dosage |
| Component | tablet | form 1 | form 2 | form 3 |
| Aliskiren hemi-fumarate | 34.53 | 55.25 | 55.25 | 48.75 |
| Microcrystalline cellulose | 45.97 | 28.25 | 24.08 | 31.545 |
| Polyvinylpyrrolidon K 30 | — | — | 4 | 3.53 |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of aliskiren 150 mg (free base) uncoated tablets in mg/unit (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren hemi-fumarate | 165.75 | 165.75 | 165.75 | 165.75 |
| Phase | Microcrystalline cellulose | 220.65 | 84.75 | 72.25 | 90.25 |
| | Polyvinylpyrrolidon K 30 | — | — | 12.00 | 12.00 |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline cellulose | — | — | — | 17.00 |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of aliskiren 150 mg (free base) uncoated tablets in % by weight (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren hemi-fumarate | 34.53 | 55.25 | 55.25 | 48.75 |
| Phase | Microcrystalline cellulose | 45.97 | 28.25 | 24.08 | 26.545 |
| | Polyvinylpyrrolidon K 30 | — | — | 4 | 3.530 |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline cellulose | — | — | — | 5 |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### EXAMPLE 2

Composition of aliskiren (dosage form 3) film-coated tablets in mg/unit.

| | Dosage form 3/Strength | | |
|---|---|---|---|
| | 75 mg | 150 mg | 300 mg |
| Component | (free base) | (free base) | (free base) |
| Aliskiren hemi-fumarate | 82.875 | 165.750 | 331.500 |
| Microcrystalline cellulose | 53.625 | 107.250 | 214.500 |
| Polyvinylpyrrolidon K 30 | 6.000 | 12.000 | 24.000 |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated tablet | 180.000 | 357.000 | 706.000 |
| weight | | | |

The dosages forms 1, 2 and 3 may be prepared, e.g., as follows:
1) mixing the active ingredient and additives and granulating said components with a granulation liquid;
2) drying a resulting granulate;
3) mixing the dried granulate with outer phase excipients;
4) compressing a resulting mixture to form a solid oral dosage as a core tablet; and
5) optionally coating a resulting core tablet to give a film-coated tablet.

The granulation liquid can be ethanol, a mixture of ethanol and water, a mixture of ethanol, water and isopropanol, or a solution of polyvinylpyrrolidones (PVP) in the before mentioned mixtures. A preferred mixture of ethanol and water ranges from about 50/50 to about 99/1 (% w/w), most preferrably it is about 94/6 (% w/w). A preferred mixture of ethanol, water and isopropanol ranges from about 45/45/5 to about 98/1/1 (% w/w/w), most preferably from about 88.5/5.5/6.0 to about 91.5/4.5/4.0 (% w/w/w). A preferred concentration of PVP in the above named mixtures ranges from about 5 to about 30% by weight, preferably from about 15 to about 25%, more preferably from about 16 to about 22%.

Attention is drawn to the numerous known methods of granulating, drying and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press.

The manufacturing of the granulate can be performed on standard equipment suitable for organic granulation processes. The manufacturing of the final blend and the compression of tablets can also be performed on standard equipment.

For example, step (1) may be carried out by a high-shear granulator, e.g., Collette Gral; step (2) may be conducted in a fluid-bed dryer; step (3) may be carried out by a free-fall mixer (e.g. container blender, tumble blender); and step (4) may be carried out using a dry compression method, e.g., a rotary tablet press.

Although the present invention has been described in considerable detail with reference to certain preferred versions thereof, other versions are possible without departing from the spirit and scope of the preferred versions contained herein. All references and patents (U.S. and others) referred to herein are hereby incorporated by reference in their entirety as if set forth herein in full.

1. A combination comprising
i) a therapeutic agent acting on the renin-angiotensin system (RAS) or a pharmaceutically acceptable salt thereof, and
ii) at least one CB1 antagonist, or a pharmaceutically acceptable salt thereof.

2. A combination according to claim 1 wherein the therapeutic agent acting on the renin-angiotensin system (RAS) is selected from the group consisting of a renin inhibitor, an angiotensin II receptor blocker (ARB) and an angiotensin converting enzyme (ACE) inhibitor.

3. A Combination according to claim 1 wherein the therapeutic agent acting on the renin-angiotensin system (RAS) is a renin inhibitor, preferably selected from the group consisting of RO 66-1132, RO 66-1168 and a compound of the formula 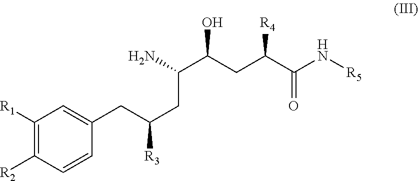
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

4. A combination according to claim 3 wherein the renin inhibitor is a compound of formula (III) having the formula 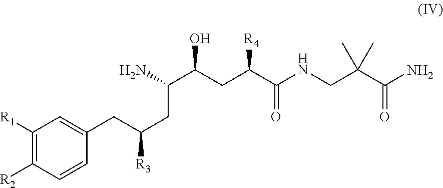
wherein R₁ is 3-methoxypropyloxy; R₂ is methoxy; and R₃ and R₄ are isopropyl; or a pharmaceutically acceptable salt thereof.

5. A combination according to claim 1 wherein the wherein the therapeutic agent acting on the renin-angiotensin system (RAS) is an angiotensin II receptor blocker (ARB), preferably selected from the group consisting of valsartan, losartan, candesartan, eprosartan, irbesartan, olmesartan, tasosartan, telmisartan, the compound with the designation E-4177 of the formula 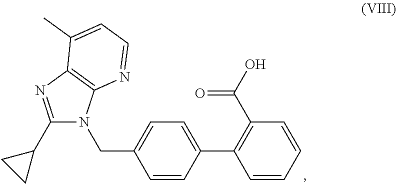
the compound with the designation SC-52458 of the following formula 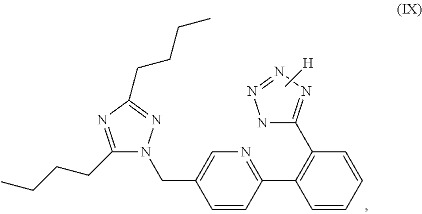
and the compound with the designation the compound ZD-8731 of the formula 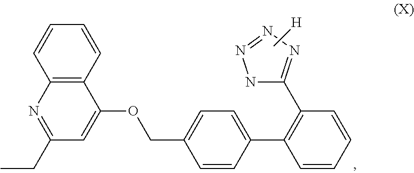
or, in each case, a pharmaceutically acceptable salt thereof.

6. (canceled)

7. A combination according to claim 1 wherein the wherein the therapeutic agent acting on the renin-angiotensin system (RAS) is an angiotensin converting enzyme (ACE) inhibitor, preferably selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril or a pharmaceutically acceptable salt thereof.

8. (canceled)

9. A combination according to claim 1, wherein the CB1 antagonist is selected from the group consisting of rimonabant, AM-251 and SR147778 or, in each case, a pharmaceutically acceptable salt thereof.

10-14. (canceled)

15. A combination according to claim 1, wherein
i) aliskiren is administered in an amount of from 50 to 500 mg daily, and
ii) rimonabant is administered in an amount between 5 and 40 mg or between 5 and 20 mg daily,
or in any case or a pharmaceutically acceptable salt thereof.

16. A combination according to claim 1, wherein
i) 75, 150 or 300 mg of aliskiren is administered daily, and
ii) 5, 10 or 20 mg of rimonabant is administered daily,
or in any case or a pharmaceutically acceptable salt thereof.

17. A combination according to claim 1, further comprising at least one additional pharmaceutically acceptable carrier.

18. A combination according to claim 1, in the form of a combined preparation or a fixed combination.

19. A method for the manufacture of a medicament for the prevention, delay of progression or treatment of diseases and disorders that may be modulated by action on the renin-angiotensin system (RAS), obesity, appetency disorders or substance abuse disorders comprising mixing i) and ii) of claim 1 with additives and granulating said components with a granulation liquid;
drying a resulting granulate;
mixing the dried granulate with outer phase excipients;
compressing a resulting mixture to form a solid oral dosage as a core tablet; and
optionally coating a resulting core tablet to give a film-coated tablet.

20. The method according to claim 19, wherein the disease or disorder is selected from the group consisting of:
(a) hypertension, congestive heart failure, renal failure, especially chronic renal failure, restenosis after percutaneous transluminal angioplasty, and restenosis after coronary artery bypass surgery;
(b) atherosclerosis, e.g., due to a reduction in oxidant stress, a direct effect on lipids or to an anti-inflammatory effect of one or all components of the combination;
(c) insulin resistance and syndrome X/metabolic syndrome, diabetes mellitus type 2, obesity, nephropathy, renal failure, e.g. chronic renal failure, hypothyroidism, survival post myocardial infarction (MI), coronary heart diseases, hypertension in the elderly, familial dyslipidemic hypertension, increase in the formation of collagen, fibrosis, eg., cardiac, renal or liver, remodeling (vascular) following hypertension and/or hyperlipidemia (antiproliferative effect of the combination which may be dependent or independent of an action on lipids), and vascular remodeling which may be, in part, due to an anti-inflammatory effect and all these diseases or conditions associated with or without hypertension;
(d) endothelial dysfunction with or without hypertension;
(e) hyperlipidemia, hyperlipoproteinemia, atherosclerosis and hypercholesterolemia;
(f) glaucoma;
(g) isolated systolic hypertension (ISH);
(h) diabetic retinopathy;
(i) peripheral vascular disease;
(j) obesity;
(k) appetency disorders:
(l) substance abuse disorders; and
(j) increased appetite associated with nicotine or tobacco withdrawal.

21-22. (canceled)

23. The method according to claim 19, for body fat reduction.

24. A method for the prevention of, delay of progression of, treatment of diseases and disorders that may be modulated by action on the renin-angiotensin system (RAS), obesity, appetency disorders or substance abuse disorders, comprising administering to a warm-blooded animal, including man, in need thereof an effective amount of the combination according to claim 1.

25. A method according to claim 24, wherein the of disease or disorder that may be modulated by action on the renin-angiotensin system (RAS) is selected from the group consisting of:
(a) hypertension, congestive heart failure, renal failure, especially chronic renal failure, restenosis after percutaneous transluminal angioplasty, and restenosis after coronary artery bypass surgery;
(b) atherosclerosis, eg., due to a reduction in oxidant stress, a direct effect on lipids or to an anti-inflammatory effect of one or all components of the combination;
(c) insulin resistance and syndrome X/metabolic syndrome, diabetes mellitus type 2, obesity, nephropathy, renal failure, e.g. chronic renal failure, hypothyroidism, survival post myocardial infarction (MI), coronary heart diseases, hypertension in the elderly, familial dyslipidemic hypertension, increase in the formation of collagen, fibrosis, eg., cardiac, renal or liver, remodeling (vascular) following hypertension and/or hyperlipidemia (antiproliferative effect of the combination which may be dependent or independent of an action on lipids), and vascular remodeling which may be, in part, due to an anti-inflammatory effect and all these diseases or conditions associated with or without hypertension;
(d) endothelial dysfunction with or without hypertension;
(e) hyperlipidemia, hyperlipoproteinemia, atherosclerosis and hypercholesterolemia;
(f) glaucoma;
(g) isolated systolic hypertension (ISH);
(h) diabetic retinopathy; and
(i) peripheral vascular disease;
(j) obesity;
(k) appetency disorders:
(l) substance abuse disorders; and
(m) increased appetite associated with nicotine or tobacco withdrawal.

26-27. (canceled)

28. A method according to claim 24, for body fat reduction.
